(19) United States
(12) Patent Application Publication
(10) Pub. No.: US 2013/0116673 A1
(43) Pub. Date: May 9, 2013

(54) Microscope Magnification Sensor

(71) Applicants: Cary Spediacci, Daly City, CA (US); David Hindi, San Jose, CA (US); Mathew Clopp, Santa Clara, CA (US); Bill Fish, San Jose, CA (US)

(72) Inventors: Cary Spediacci, Daly City, CA (US); David Hindi, San Jose, CA (US); Mathew Clopp, Santa Clara, CA (US); Bill Fish, San Jose, CA (US)

(73) Assignee: AMO Manufacturing USA, LLC, Santa Clara, CA (US)

(21) Appl. No.: 13/459,691

(22) Filed: Apr. 30, 2012

Related U.S. Application Data
(63) Continuation of application No. 10/703,195, filed on Nov. 5, 2003, now abandoned.

(51) Int. Cl. A61F 9/008

(52) U.S. Cl. CPC A61F 9/00804
USPC 606/5

(57) ABSTRACT

Devices, systems and methods for scaling the size and/or position of a marker on a magnified image of an object. In preferred embodiments, the object is an eye that is undergoing laser eye surgery. The eye is viewed through a magnification system or microscope and an image of the eye is presented on a display. One or more markers are present on the image, each identifying a specific target location or landmark on the eye. When a desired magnification setting is selected, the image is scaled accordingly. In addition, one or more of the markers is scaled in size and/or position to reflect the magnification setting. This allows the marker to maintain identification of the target location while reflecting the selected magnification level.

### CROSS-REFERENCES TO RELATED APPLICATIONS

The present application is a Continuation of U.S. application Ser. No. 10/703,195 filed Nov. 5, 2003. The full disclosure of which is incorporated herein by reference in its entirety for all purposes.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

The present invention is generally related to magnification systems, particularly sensing magnification settings for use in a visual display of the magnified image, especially for use in laser eye surgery systems.

Laser eye surgery is performed by a laser after optically aligning the laser with the eye using a magnification system or microscope. While it may be possible to make use of lasers having other wavelengths, known laser eye surgery procedures generally include an ultra-violet or modified frequency infrared laser to remove a microscopic layer of stromal tissue from the eye's cornea to change the cornea's contour for varying purposes, such as for correcting myopia, hyperopia, astigmatism and the like. Laser ablation results in photodecomposition of the corneal tissue, but generally does not cause significant thermal damage to adjacent and underlying tissues of the eye. The irradiated molecules are broken into smaller volatile fragments photochemically, directly breaking the intermolecular bonds.

Most laser eye systems include a microscope to aid the surgeon in aligning the patient's cornea with the laser system, and to allow the surgeon to optically monitor or verify that the targeted portion of the stroma is removed as intended. Generally, the surgeon observes the patient's eye at low magnification to orient the procedure and at progressively higher magnification to provide great resolution for finer and more accurate procedures. Known laser eye surgery systems have generally included fairly standard microscope structures. The microscope optics are typically designed to provide flat field, anastigmatic, achromatic, nearly diffraction limited imaging with optical magnification zoomable approximately over a 15-fold range of, say 15×-200×. The magnification is adjustable and is typically selected to correspond to the largest magnification which can still be comfortably used for situating a lesion (that is, the smallest field of view which can be used when magnified across the fixed display size of the video monitor). For example, for corneal refractive surgery, where the surgeon needs to observe the cornea from limbus to limbus, this corresponds to a field of view of approximately 12 to 14 mm. At the screen, the zoom optics allow for adjustable magnification in the range of about 15×-200×, for example. This enables the surgeon to view a very narrow field, on the order of a millimeter in width, or a much wider field at lesser magnification. This is useful in enabling the surgeon to assure himself that he is aimed and focused at a particular desired region.

The ability to track or follow movements of a patient's eye is recognized as a desirable feature in laser eye surgery systems. Movements of the eye include both voluntary movements and involuntary movements. In other words, even when the patient is holding “steady” fixation on a visual target, eye movement still occurs. Tracking of the eye during laser eye surgery has been proposed to avoid uncomfortable structures which attempt to achieve total immobilization of the eye. Tracking by following the subject eye tissue, i.e., recognizing new locations of the same tissue and readjusting the imaging system and the surgical laser aim to the new location, assures that the laser, when firing through a prescribed pattern, will not deviate from the pattern an unacceptable distance. Sometimes this distance is held within 5 pm throughout ophthalmic surgery, which sets a margin of error for the procedure. However, either more stringent or alternatively more lax displacement error tolerances may be desirable to improve overall system performance.

Typically at the start of the procedure, the target region of the eye is aligned with a marker, such as a fixed reticle, visible through the microscope. In some laser eye surgery systems, an image of the target region and the fixed reticle as seen through the microscope is displayed on a system monitor for viewing by the surgeon. As the eye moves during the procedure, the target region deviates in relation to the fixed reticle. To identify the target tissue and its position in relation to the fixed reticle, another marker, such as a moving cross-hair, is provided in the monitor display which indicates the tracked target tissue. Both the fixed reticle and the moving cross-hair are visible on the display unless the eye moves excessively, outside of the range of the microscope. This typically indicates that the eye is beyond the tracking range and the ablation pattern has been interrupted until the eye returns to a position within acceptable range. Thus, the reticle and cross-hair allow the surgeon to observe the eye movements of the patient throughout the procedure and identify any excessive movements of the eye.

When the surgeon changes the magnification setting of the microscope, the image of the eye displayed on the monitor is scaled accordingly. It would be desirable to provide appropriately scaled and positioned markers at any magnification setting to maintain relational information for the viewer. Such systems should be adaptable to existing laser eye surgery systems, easy to use and cost effective. At least some of these objectives will be met by the inventions described hereinafter.

### BRIEF SUMMARY OF THE INVENTION

The present invention provides devices, systems and methods for scaling the size and/or position of a marker on a magnified image of an object. In preferred embodiments, the object is an eye that is undergoing laser eye surgery. The eye is viewed through a magnification system or microscope and an image of the eye is presented on a display. One or more markers are present on the image, each identifying a specific target location or landmark on the eye. When a desired magnification setting is selected, the image is scaled accordingly. In addition, one or more of the markers is scaled in size and/or position to reflect the magnification setting. This allows the marker to maintain identification of the target location while reflecting the selected magnification level.

In a first aspect, the present invention provides a system for scaling the size and/or position of a marker on a magnified image of an object. In preferred embodiments, the system includes a magnification system that magnifies a view of the object. The magnification system includes a magnification selector that selects a magnification level for viewing of the object upon actuation, wherein selecting comprises changing from a first magnification level to a second magnification level. Thus, the magnification selector may be comprised of a knob, button, lever, switch or other mechanism which is used to select a desired magnification level. Actuation of the magnification selector changes the magnification level to the desired or selected magnification level. It may be appreciated that the magnification level may be any level from zero to any maximum level. A zero magnification level would provide an image as seen in plain view, without magnification. Thus, the magnification selector can change the magnification between plain view and magnified view and between various degrees of magnification. Example magnification levels include 1.0, 0.63, 1.6, 2.5 and 4.0. In preferred embodiments, the magnification system also includes a magnification sensor system which senses the selected magnification level. The sensor system is used to scale and position the markers as will be described in later sections.

The markers may be any of any form and may be used to convey any information to the viewer. As mentioned, in preferred embodiments, each of the markers identifies on the image a specific target location or landmark of the eye. For example, a target location or landmark may be a pupil, particularly the center of the pupil. This target location is particularly useful when tracking an eye for laser eye surgery. Often, the center of the pupil is aligned with a fixed marker, for example a fixed reticle, at the start of a surgical procedure. As the eye moves during the procedure, the pupil center deviates in relation to the fixed reticle. To identify the center of the pupil as it moves and its position in relation to the fixed reticle, another marker, such as a moving cross-hair, is provided on the image which indicates the tracked pupil center. Thus, in some embodiments, the system includes a tracking system capable of tracking a target location on the object.

The system also includes system software which provides an image of the object at the selected magnification level and a marker, such as a cross-hair, positioned on the target location of the image as the location is tracked by the tracking system. The software adjusts the position of the cross-hair based on the sensed magnification level to maintain positioning on the target location of the image when changing magnification levels. Thus, when the magnification system is set at a first magnification level, the marker is seen by the viewer on the target location of the image. When the magnification level is changed, the target location on the image may move on the display, such as along an x-axis or y-axis, due to zooming effects. The system software ensures that the cross-hair maintains positioning on the target location by an adjustment in positioning itself.

In some embodiments, the system software further adjusts the size of the cross-hair based on the sensed magnification level to maintain size relationship between the cross-hair and the image of the object when changing magnification levels. Thus, when increasing the magnification level, the cross-hair is increased in size proportionally to the magnification level and, likewise, when decreasing the magnification level, the cross-hair is proportionally decreased in size.

In addition, in some embodiments, the system software further provides another marker, such as a fixed reticle, which has a center indicating a reference position for the target location. The software may adjust the size of at least the center of the fixed reticle based on the sensed magnification level to maintain size relationship between the center of the fixed reticle and the image of the object when changing magnification levels. Thus, the cross-hair and the fixed reticle may be of the same size or differing sizes. But, in either case, each or both of the markers may be scaled with the magnification level.

As mentioned, the magnification system also includes a magnification sensor system which senses the selected magnification level. The sensor system is used to scale and position the markers. In preferred embodiments, the magnification sensor system generates a gray scale code which signifies the selected magnification level. The magnification sensor system may include a gray scale encoder and at least one opto-sensor which is able to sense a portion of the encoder to generate the gray scale code. The gray scale encoder may have the shape of a wheel wherein the portion sensed by the opto-sensors is a lip which protrudes around a portion of the wheel. The gray scale encoder wheel is rotated by selection of the magnification level, such as by rotation of a knob which selects a magnification level at each rotation stop or detent. At each rotation stop, the opto-sensors each sense the presence or absence of the lip of the gray scale encoder. The presence of the lip encodes a “1” and the absence of the lip encodes a “0”. Together, the codes from the opto-sensors generates a gray scale code. In preferred embodiments, the at least one opto-sensor comprises three opto-sensors which together generate the gray scale code. Rotation of the wheel to the next rotation stop rotates the lip, thereby generating a different gray scale code. In some embodiments, three opto-sensors generate gray scale codes corresponding to six magnification levels or rotation stops.

In a second aspect, the present invention provides a method for scaling the size and/or position of a marker on a magnified image of an object. In preferred embodiments, the method includes providing a magnification system which magnifies a view of an object to a desired magnification level upon selection of the desired magnification level. The method also includes selecting the desired magnification level by actuating a magnification selector which changes the view from an existing magnification level to the selected magnification level, the selecting step actuating a magnification sensor which senses the selected magnification level. Typically, the method also includes tracking a target location on the object with a tracking system. An image of the object is then viewed at the selected magnification level and a marker positioned on the target location of the image as the location is tracked by the tracking system, wherein the position of the marker has been adjusted based on the sensed magnification level to maintain positioning on the target location of the image when changing from the existing magnification level to the selected magnification level.

In some embodiments, the size of the marker has additionally been adjusted based on the sensed magnification level to maintain size relationship between the marker and the image of the object when changing from the existing magnification level to the selected magnification level.

In additional embodiments, the viewing step further includes viewing an additional marker, such as a fixed reticle, having a center indicating a reference position for the target location, wherein the size of at least the center of the fixed reticle has been adjusted based on the sensed magnification level to maintain size relationship between the at least the center of the fixed reticle and the image of the object when changing from the existing magnification level to the selected magnification level. It may be appreciated that the markers may each be of the same or different size at a selected magnification level. And, the magnification levels may be of any magnification, including zero magnification or plain view.

When the magnification system comprises a microscope adapted for use in laser eye surgery, the object is a patient's eye. Thus, the method may also include ablating the patient's eye with the use of a laser eye surgery system while viewing the image of the eye. The patient's eye includes a pupil having a center, and typically the target location for placement of a marker is the pupil center on the image. In this case the viewing step may further include viewing the marker, such as a cross-hair, on the pupil center of the image of the patient's eye. Thus, as the pupil center moves and is tracked by the tracking system, the viewer may follow the tracked movements of the pupil center by watching the moving cross-hair on the visual display.

Actuation of the magnification sensor generates a gray scale code which signifies the selected magnification level. In preferred embodiments, selecting the desired magnification level manipulates a gray scale encoder so that at least one opto-sensor changes its ability to sense a portion of the encoder which generates the gray scale code. Manipulation of the gray scale encoder may include rotation of the gray scale encoder, such as by a knob which is used to change the magnification level. The method may further include selecting another desired magnification level by actuating the magnification selector. This in turn would generate another gray scale code by the magnification sensor. In some embodiments, the method includes selecting up to six desired magnification levels by actuating the magnification selector. The generated gray scale codes used to scale and position the markers as described above.

Other objects and advantages of the present invention will become apparent from the detailed description to follow, together with the accompanying drawings.

### BRIEF DESCRIPTION OF THE DRAWINGS

FIG. 1 illustrates a laser eye surgery system of the present invention including a magnification system through which an eye of a patient is viewed.

FIGS. 2A-2C illustrate a display showing images of the patient's eye and at least one marker.

FIG. 3 provides a schematic illustration of a portion of the microscope of the laser eye surgery system.

FIG. 4 provides a table of magnification settings.

FIG. 5 illustrates an embodiment of the magnification setting sensor system.

FIG. 6 provides a table of pin-outs for a DB9M connection.

FIG. 7 illustrates a gray scale encoder engaged with a PCB.

FIG. 8 provides a table which shows the detent positions of the knob with corresponding detent degree positions, corresponding gray scale codes and corresponding magnification settings.

FIGS. 9A-9D illustrate the generation of the gray scale code by the magnification setting sensor system.

FIG. 10 illustrates the sensor system disposed in the microscope body.

### DETAILED DESCRIPTION OF THE INVENTION

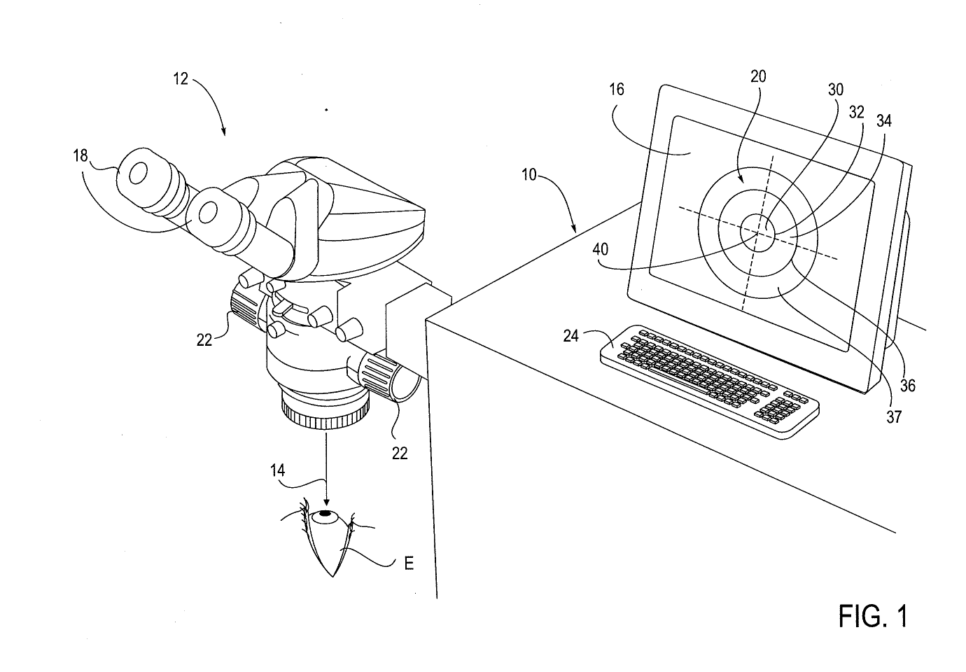

Referring now to FIG. 1, an embodiment of a laser eye surgery system 10 of the present invention includes a magnification system or microscope 12 through which an eye E of a patient is viewed, typically while the eye E is ablated by a laser beam 14. In preferred embodiments, the microscope 12 comprises a Leica MS5 microscope, however any suitable microscope or microscope components may be used. The eye E may be viewed by a surgeon through eyepieces 18 on the microscope 12. The microscope 12 includes, among other components, knobs 22 for adjusting the magnification of the microscope 12. Thus, by rotation of the knobs 22, the eye E may be viewed under varying levels of magnification through the eyepieces 18. In addition, laser eye surgery system 10 of the present invention includes a monitor display 16 which provides an image 20 of the eye E as viewed through the eye pieces 18. This allows the surgeon and any other assistants or practitioners to easily view the eye E throughout the surgical procedure without approaching the eyepieces 18 of the microscope 12. The display 16 may also provide additional information related to the procedure and provide a user interface with the use of a keyboard 24 for user input.

The image 20 of the eye E may include a pupil image 30 having a pupil/iris boundary 32, an iris image 34 having a limbus 36, and a sclera image 37 as shown on the display 16 of FIG. 1. At the start of the surgical procedure, the eye E is aligned with the desired path of the laser beam 14 by centering a target tissue area of the eye E with the center of a fixed reticle 40. The fixed reticle 40 may be viewed through the eyepieces 18 and is also projected into the image 20 for viewing on the display 16. In this example, the pupil image 30 is centered to be aligned with the center of the fixed reticle 40.

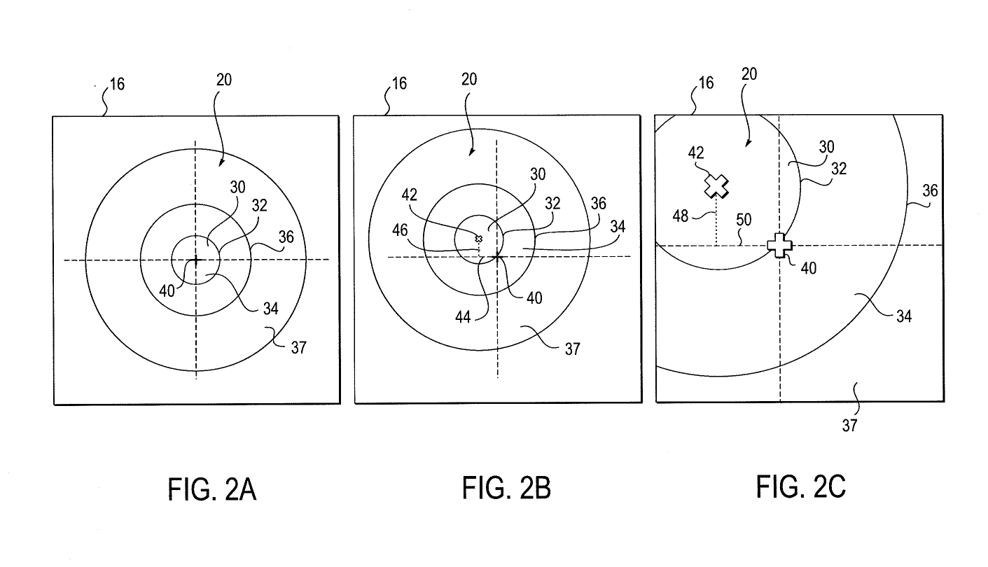

FIG. 2A provides a closer view of the display 16 of FIG. 1 having the pupil image 30 aligned with the center of the fixed reticle 40. As mentioned, the eye E typically moves both voluntarily and involuntarily while the eye E is generally aligned. Such movement is tracked by an eye tracker. Tracking by following the subject eye tissue, i.e., recognizing new locations of the same tissue and readjusting the imaging system and the surgical laser aim to the new location, assures that the laser, when firing through a prescribed ablation pattern, will not deviate from the pattern an unacceptable distance. FIG. 2B illustrates the eye E having moved away from its previous position during tracking. Software included in the system 10 projects onto the display 16 a moving cross-hair 42 which tracks the previously centered eye tissue (in this case the center of the pupil). As shown, the moving cross-hair 42 may be displaced from the fixed reticle center 40 by an x-distance 44 along an x-axis and a y-distance 46 along a perpendicular y-axis. In addition, the moving cross-hair 42 may rotate in relation to the fixed reticle center 40, as shown.

By rotation of the knobs 22, the eye E may be viewed under varying levels of magnification. As the magnification level is changed, the image 20 on the display 16 is appropriately scaled to show the image 20 at the new magnification level. In addition, the fixed reticle 40 and the moving cross-hair 42 are also scaled in size and position to reflect the new magnification level. FIG. 2C illustrates the image 20 of FIG. 2B at a higher level of magnification. As shown, the size of the reticle 40 and cross-hair 42 are appropriately larger and the cross-hair 42 is displaced by x′-distance 50 along the x-axis and y′-distance 48 along the perpendicular y-axis, wherein x′ and y′ are scaled to reflect the magnification level. Thus, the cross-hair 42 maintains representation of tracking the previously centered eye tissue (in this case the center of the pupil). Such rescaling is achieved with software of the laser eye surgery system 10.

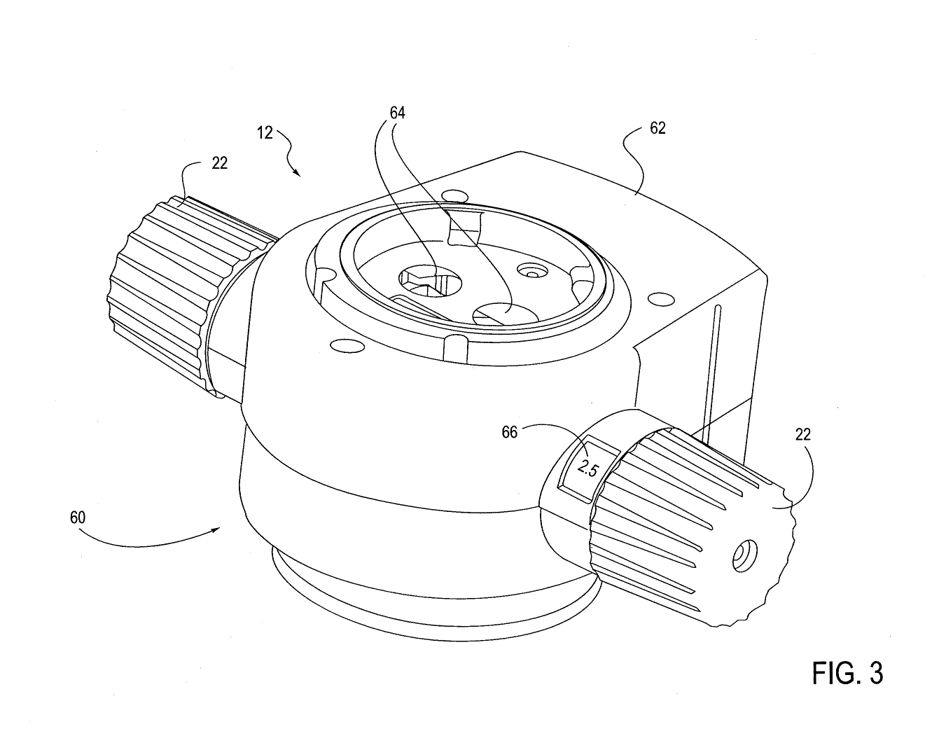

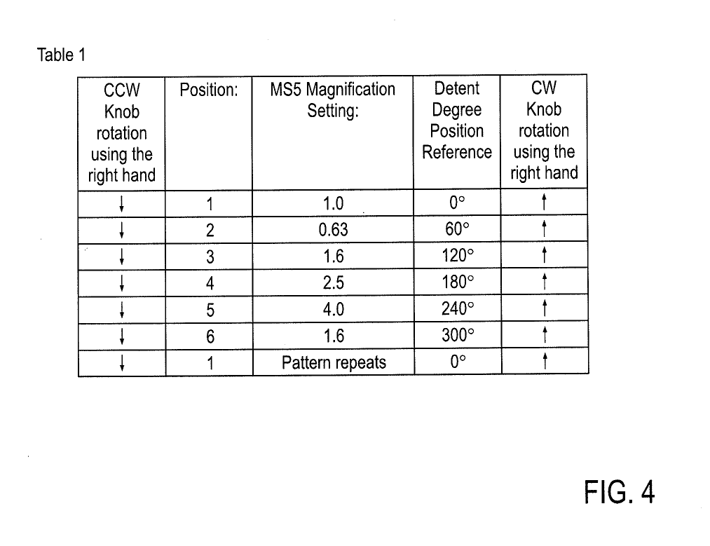

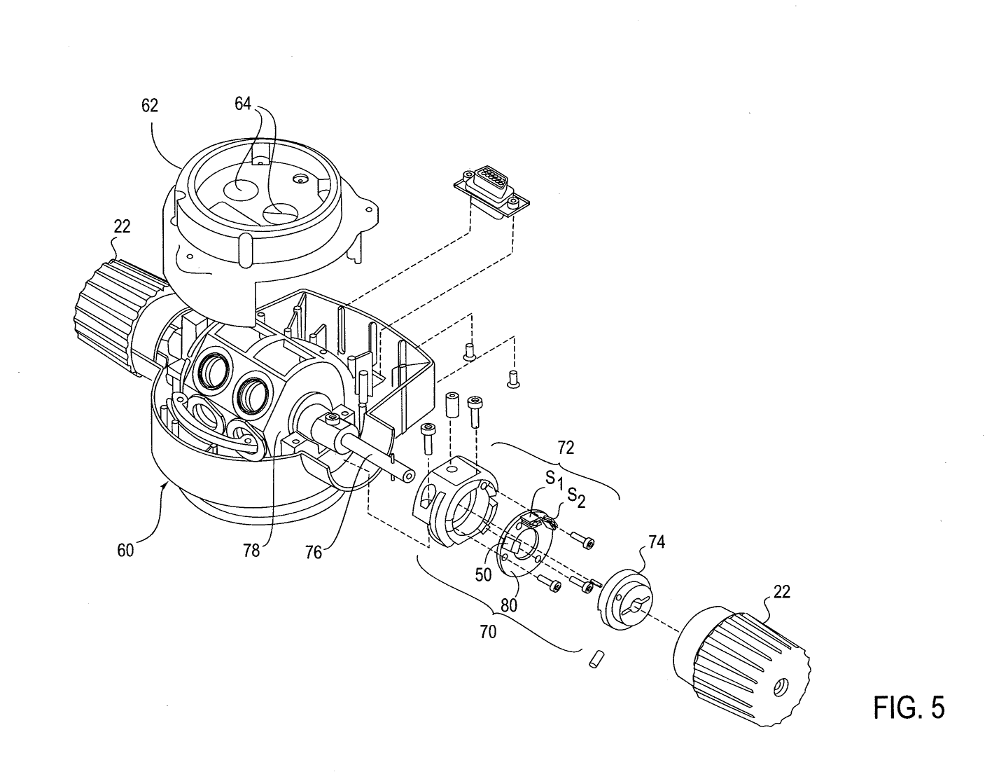

FIG. 3 provides a schematic illustration of a portion of the microscope 12 of the laser eye surgery system 10. This portion includes a microscope body 60 having a microscope top 62. Viewholes 64 are visible through the microscope top 62 which allow viewing through the eyepieces (not shown) and the microscope body 60. In addition, the portion includes knobs 22 for adjusting the magnification level. Typically, a magnification setting indicator 66 is present to display the magnification level. In this illustration, an indicator 66 is positioned near the rotating knob 22 displaying the level “2.5”. It may be appreciated that the indicator 66 may be present at any location(s) including the display 16. The Leica MS5 microscope has six magnification position settings. The six positions are continuous wherein the magnification knob has no 360 degree rotation stops, the knob 22 rotated beyond 360 degrees repeats the six magnification settings. Table 1 provided in FIG. 4 shows the possible magnification settings of this embodiment.

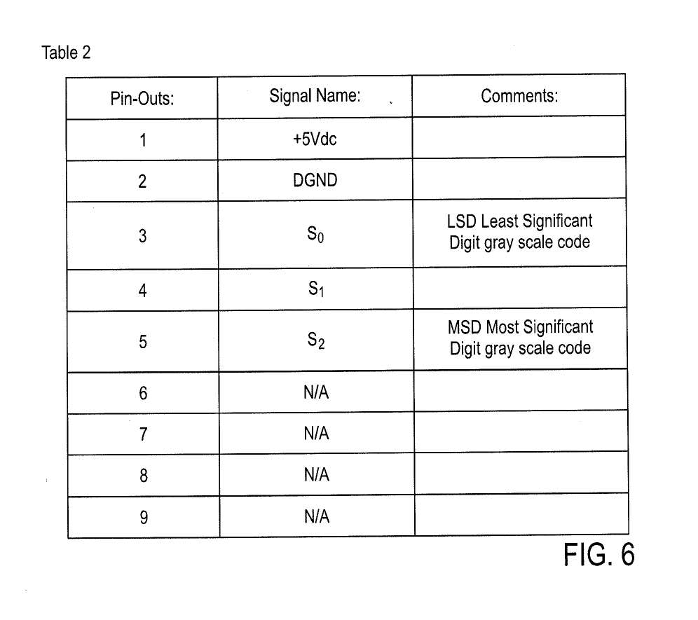

The present invention includes a magnification setting sensor system which informs the laser eye surgery system software of the magnification setting. FIG. 5 illustrates an embodiment of the magnification setting sensor system 70. The sensor system 70 comprises a magnification sensor body 72 including at least one opto-sensor, in this embodiment three opt-sensors (S₀, S₁, S₂) are present. The opto-sensors S₀, S₁, S₂ are disposed on a Printed Circuit Board (PCB) 80 with a cable connection to the rear of the magnification sensor body 72. The cable connection is DB9M; the pin-outs for the DB9M connection are presented in Table 2 of FIG. 6. The sensor system 70 also includes a gray scale encoder 74. The encoder 74 is positioned between the magnification sensor body 72 and the knob 22. The encoder 74 and knob 22 are attached to a knob shaft 76 so that rotation of the knob 22 rotates the encoder 74 in addition to rotating a magnification carousel 78 which provides lenses to magnify the viewed object, in this case the eye E.

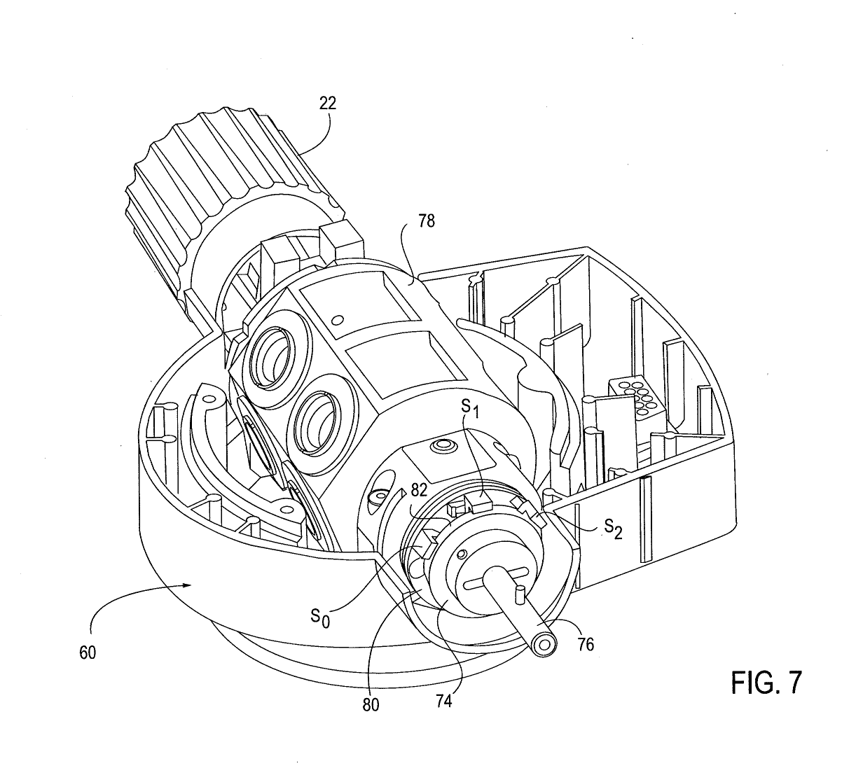

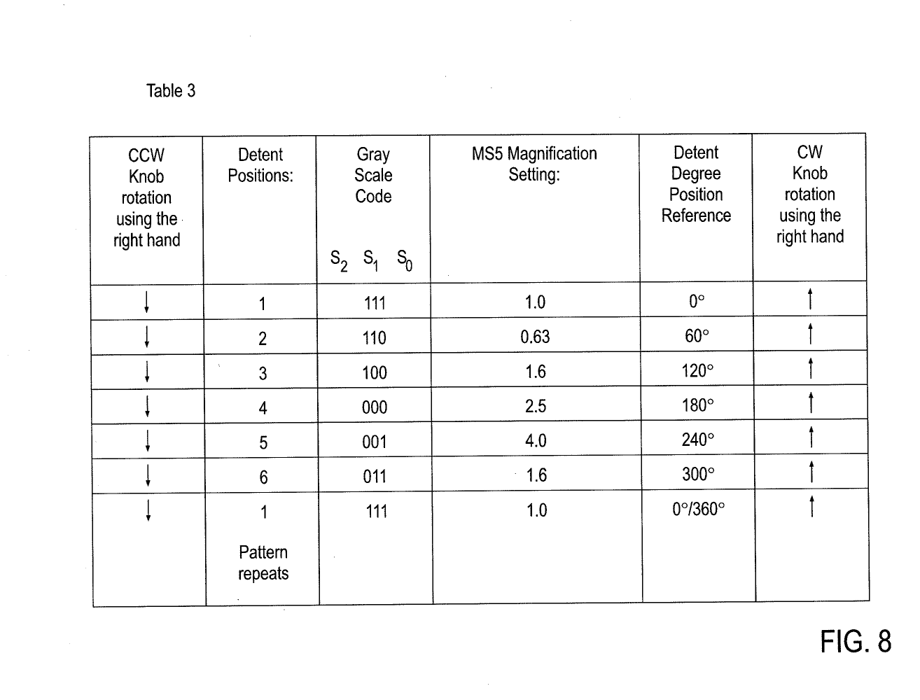

FIG. 7 illustrates the gray scale encoder 74 engaged with the PCB 80. As shown, the opto-sensors S₀, S₁, S₂ are shaped to extend over a lip 82 on the encoder 74. When the encoder 74 is rotated through the magnification settings or rotation stops by rotation of the knob 22, the lip 82 is also rotated through the rotation stops. Since the lip 82 extends only along a portion of the encoder 74, in this embodiment along 180 degrees of the encoder 74, the presence of the lip 82 is sensed by the sensors S₀, S₁, S₂ as the encoder 74 is rotated in either the clockwise or counter-clockwise direction. Sensing of the presence of the lip 82 of the gray scale encoder 74 provides a gray scale code which indicates the rotation stop that the knob 22 has been turned to, which in turn indicates the magnification setting of the microscope. The gray scale code is a variation of the standard binary code in which only one bit changes at a time between successive binary digits. FIG. 8 provides Table 3 which shows the detent positions of the knob 22 with corresponding detent degree positions, corresponding gray scale codes and corresponding magnification settings.

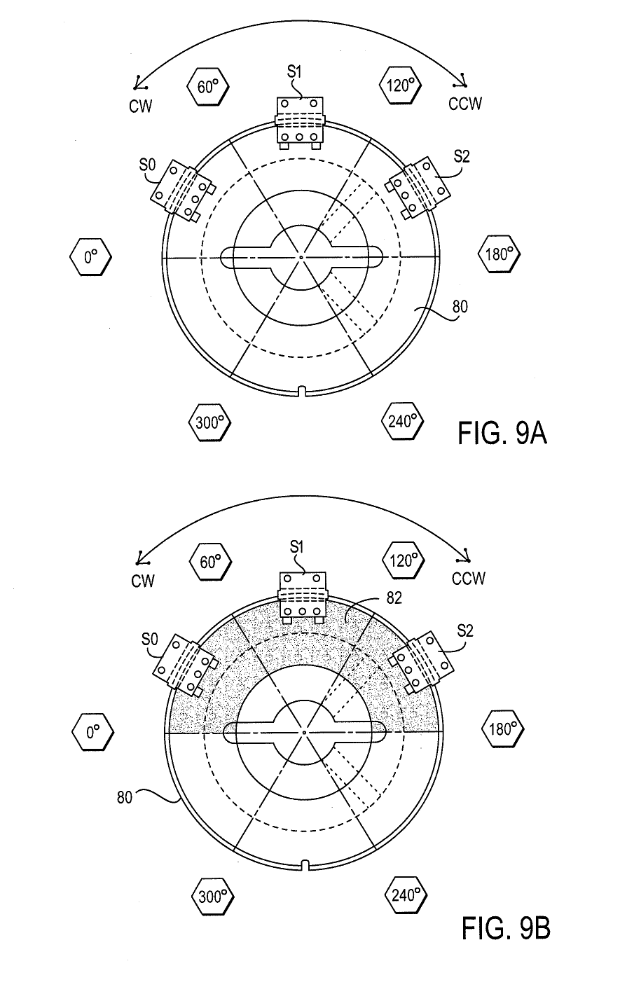

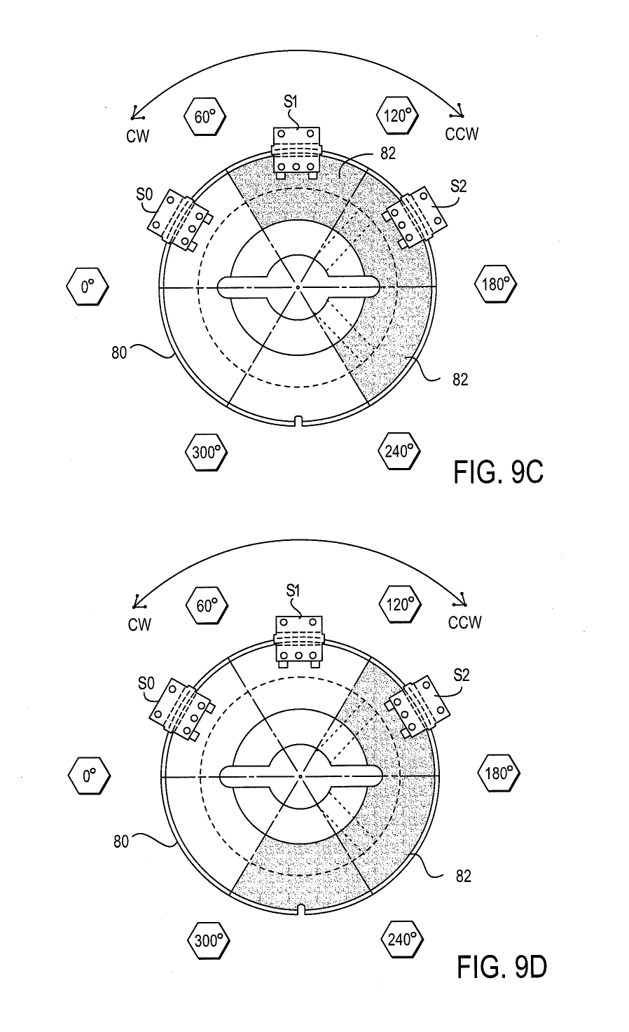

FIGS. 9A-9D further illustrate the generation of the gray scale code by the magnification setting sensor system 70. FIG. 9A illustrates the view from the PCB circuit side looking through the PCB back at the gray scale encoder. In this embodiment, the opto-sensors S₀, S₁, S₂ are disposed at the 30, 90, 150 degree positions respectively. The opto- sensors remain stationary as the gray scale encoder rotates. FIG. 9A also illustrates the locations of the six detents disposed 60 degrees apart, at 0, 60, 120, 180, 240 and 300 degrees. FIGS. 9B-9D illustrate the movement of the lip 82 (indicated by shading) as the gray scale encoder 74 is rotated through the detents. FIG. 9B illustrates the encoder 74 at the first detent wherein the lip 82 is sensed by all three opto-sensors S₀, S₁, S₂. This provides a gray scale code of 111. The gray scale code is transmitted from the PCB 80 to the system controller of the laser eye surgery system 10. The system controller software determines if valid magnification position settings are being sent from the PCB 80. Referring to Table 3 of FIG. 8, a gray scale code of 111 corresponds to a magnification setting of 1.0. Thus, the system software appropriately scales the size and position of the reticle 40 and cross-hair 42 on the image 20, as described and illustrated in FIGS. 2A-2C, to reflect the magnification setting of 1.0. In addition, the system software may display the sensed magnification setting.

FIG. 9C illustrates rotation of the encoder 74 counter clockwise (CCW) to the second detent wherein the lip 82 is rotated so that the lip 82 is sensed by opto-sensors S₁, S₂. This provides a gray scale code of 110. Referring to Table 3 of FIG. 8, a gray scale code of 110 corresponds to a magnification setting of 0.63. Thus, the system software appropriately scales the size and position of the reticle 40 and cross-hair 42 on the image 20, as described and illustrated in FIGS. 2A-2C, to reflect the magnification setting of 0.63. In addition, the system software may display the sensed magnification setting.

FIG. 9D illustrates rotation of the encoder 74 counter clockwise (CCW) to the third detent wherein the lip 82 is rotated so that the lip 82 is sensed by opto-sensor S₂. This provides a gray scale code of 100. Referring to Table 3 of FIG. 8, a gray scale code of 100 corresponds to a magnification setting of 1.6. Thus, the system software appropriately scales the size and position of the reticle 40 and cross-hair 42 on the image 20, as described and illustrated in FIGS. 2A-2C, to reflect the magnification setting of 1.6. In addition, the system software may display the sensed magnification setting. It may be appreciated that rotation of the encoder 74 through the remaining detent positions will continue rotating the lip 82 and providing gray scale codes in the same manner. In this way, the magnification settings are transmitted through the system software to be used in conjunction with the display.

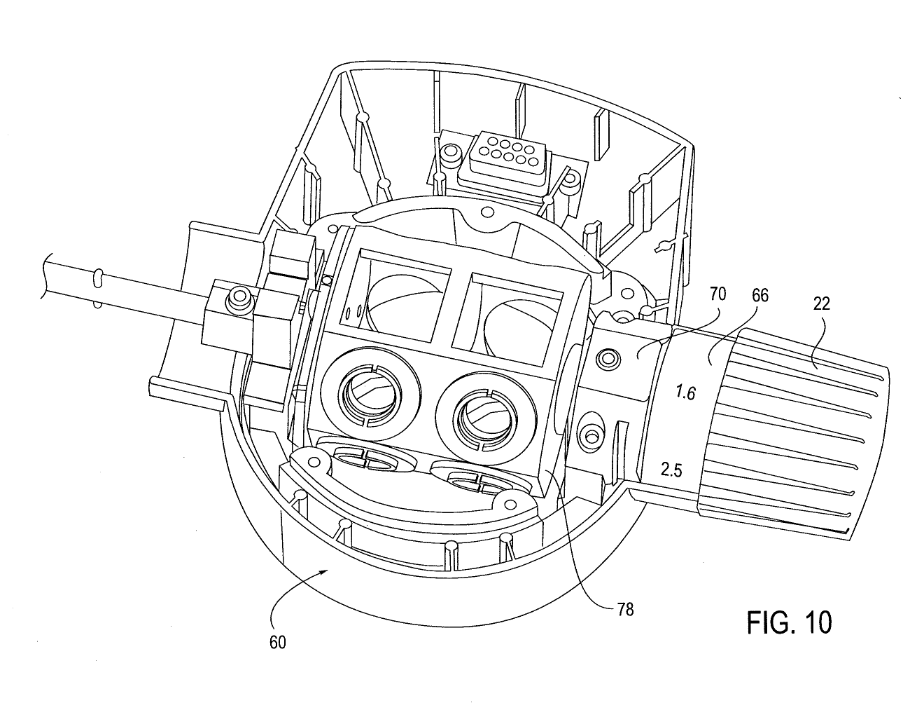

The magnification setting sensor system 70 is a compact system which is easily incorporated into existing microscopes. As shown in FIG. 10, the sensor system 70 is disposed within the microscope body 60, between the magnification carousel 78 and the knob 22. Thus, the knob 22 and magnification setting indicator 66 may appear identical to a standard microscope so that the magnification setting sensor system 70 is an unobtrusive addition to the laser eye surgery system 10.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. A system comprising:
a magnification system which magnifies a view of an object including
a magnification selector that selects a magnification level for viewing of the object upon actuation, wherein selecting comprises changing from a first magnification level to a second magnification level, and
a magnification sensor system which senses the selected magnification level;
a tracking system capable of tracking a target location on the object; and
system software which provides an image of the object at the selected magnification level and a marker positioned on the target location of the image as the location is tracked by the tracking system, wherein the software adjusts the position of the marker based on the sensed magnification level to maintain positioning on the target location of the image when changing from the first magnification level to the second magnification level.
